(19) United States
(12) Patent Application Publication
(10) Pub. No.: US 2011/0207154 A1
(43) Pub. Date: Aug. 25, 2011

(54) METHOD FOR DETERMINATION OF MARINOBUFAGENIN LEVELS AND COMPOUNDS EMPLOYABLE IN SUCH METHOD

(75) Inventors: JULES B. PUSCHETT, Temple, TX (US); Daniel Romo, College Station, TX (US); Luc R. Berghman, College Station, TX (US); Daad Ali Abi-Ghanem, College Station, TX (US); Xinzhong Lai, Edison, NJ (US)

(73) Assignee: SCOTT & WHITE HEALTHCARE, Temple, TX (US)

(21) Appl. No.: 12/974,482

(22) Filed: Dec. 21, 2010

Related U.S. Application Data
(60) Provisional application No. 61/294,272, filed on Jan. 12, 2010.

(51) Int. Cl. G01N 33/566; C07K 14/00; C07K 14/765; C07J 19/00; C07J 43/00; C07K 14/435

(52) U.S. Cl. 435/7.92; 530/410; 530/363; 540/4; 540/83; 530/409; 530/386; 436/501

(57) ABSTRACT

The present invention is directed toward a method for determination of marinobufagenin concentration in a body specimen through conjugation of marinobufagenin to a suitable protein, thereby creating a conjugate which will trigger an antibody response in a host. The conjugated marinobufagenin is immunogenic. The antibodies so produced may be employed in an ELISA test to ascertain the concentration of marinobufagenin in a body specimen. A number of unique compounds are created in the process and are disclosed. An ELISA assay may be employed.

### CROSS-REFERENCE TO RELATED APPLICATION

The present application claims the benefit of U.S. Provisional patent application Ser. No. 61/294,272, filed Jan. 12, 2010, and entitled, “METHOD FOR DETERMINATION OF MARINOBUFAGENIN LEVELS AND COMPOUNDS EMPLOYABLE IN SUCH METHOD,” which is herein incorporated by reference in its entirety.

### BACKGROUND OF THE INVENTION

1. Field of the Invention

The present invention relates to a method for determining the quantity of marinobufagenin (“MBG”) present in a body specimen, such as blood or urine, and also relates to unique compounds employable in such method.

2. Description of the Prior Art

Hypertension is a public health problem in the United States. Nearly 73 million Americans suffer from this disorder, and its incidence is rising. Fields L E, Burt V L, Cutler J A, et al., “The Burden of Adult Hypertension in the United States 1999 to 2000: A Rising Tide,” Hypertension, 2004;44:398-404. Almost one third of adults have been diagnosed with this disorder and the number of adults with hypertension increased by about 30% between the late 1980s and the early 1990s. Obesity and the aging process account for some of the increase in hypertensive patients (Cheung B M Y, Ong K L, Man Y B, et al., “Prevalence, awareness, treatment, and control of hypertension. United States National Health and Nutrition Examination Survey 2001-2002,” J. Clin. Hypertens., 2006;8:93-98), but the epidemic of diabetes, now encountered more often in teenagers, will, no doubt, continue to impact the incidence rate.

The current therapy of hypertension is based largely upon epidemiologic data. However, the basis for many treatment paradigms is the division of hypertension into two broad etiopathogenetic categories. The latter reflect the physical relationships between blood flow, blood pressure, and the resistance to flow in the vascular circuit. The expression which relates these physical elements is Q (flow)=P (pressure)/R (resistance). If one solves for pressure, one obtains the equation P=Q×R. One may perturb pressure either by altering the flow term or the resistance term or both. In mammalian species, including man, flow is a function of cardiac output (“CO”). CO, in turn, depends crucially upon venous return to the heart, and the latter is directly related to the extracellular fluid (“ECF”) volume, especially the intravascular volume. Accordingly, the flow term in the equation above can be replaced by ECF volume, leading to the recognition that there are two major pathogenetic mechanisms in the development of hypertension: 1) expansion of the ECF volume and 2) increased total peripheral resistance. If one excludes from consideration the secondary causes of hypertension, somewhere in the neighborhood of 90-95% of hypertension is characterized as “essential.” Kaplan N M, Flynn J T., “Kaplan's Clinical Hypertension, ninth edition, 2006,” Lippincott, Williams and Wilkins, Philadelphia, pp. 50-121. This term denotes the fact that specific etiologic factors remain unknown. However, it is clear that essential hypertension is a syndrome with multiple etiologies. Furthermore, if one examines the two broad etiologic categories of hypertension (1) expansion of the ECF volume and 2) increased peripheral resistance) the data suggest that the former category accounts for almost 30-40% of the “essential” hypertension, and the latter approximately 60-70%. Laragh J H, Letcher R L, Pickering T G., “Renin profiling for diagnosis and treatment of hypertension,” JAMA, 1979:41:151-156.

A prime focus of the present invention is the cause of volume expansion-mediated hypertension, which, over time, has achieved greater prominence. Current thought is that, over time, this form of hypertension may account for more than 40% of the hypertensives in the United States given the increasing number of the elderly, obese, and diabetics. Volume expansion-mediated hypertension is seen primarily in the following demographic groups: 1) African-Americans, 2) the elderly, 3) the obese, 4) a subset of Type II diabetics, and 5) Hispanics. It is anticipated that, because of the aging of the population and the increasing number of obese and diabetic persons, as well as the expected increase in the proportion of the population who are Hispanic, the 40%/160% relationship between the pathogenetic groups in essential hypertension referred to above will reverse in the next 20-30 years. In addition, although they are not essential hypertensives, it is believed that a significant number of patients with preeclampsia have, as their primary etiology, hypertension based upon excessive volume expansion.

There are urgent needs for diagnostic tests to guide the therapy of hypertension. There is also a need to develop therapeutic agents directed specifically at the underlying pathophysiology, as determined by diagnostic testing. Such a system of diagnostic/therapeutic matching would greatly improve the management of hypertensive patients, would eliminate guesswork with respect to prescriptive practices, would lead to more rapid and better control of blood pressure and, in the long run, would positively impact the ability to prevent hypertensive complications, which include heart attack and stroke. Such pretreatment testing would introduce “personalized medicine” in the management of hypertension. In addition, such developments would have a positive impact on the costs of healthcare.

Preeclampsia is an example of volume expansion-mediated hypertension. The present invention involves methods and compounds which facilitate early detection and possible treatment of preeclampsia. The present invention seeks to move toward the goal of prevention of this syndrome. Preeclampsia is a disorder that consists in the de novo development of hypertension and proteinuria after 20 weeks of gestation. The syndrome often includes excessive edema formation and intrauterine growth restriction (“IUGR”). Preeclampsia occurs in from 3%-10% of all pregnancies. Pridjian G, Puschett J B, “Preeclampsia, Part I: Clinical and pathophysiological considerations,” Obstet. Gyn. Survey, 2002;57:598-618. It is the second leading cause of fetal wastage, as well as maternal morbidity and mortality. Remarkably, the hypertension, proteinuria, and edema completely resolve within 12 weeks of parturition. The only definitive therapy of this disorder is the delivery of the fetus and placenta. The latter organ appears to be the offending agent. Unfortunately, because of the IUGR, and consequent prematurity, the fetus may not survive. Therapy for preeclampsia has not changed in over 40 years and remains unsatisfactory. Preeclampsia is of great interest because pregnancy represents nature's experiment in volume expansion. In fact, pregnant patients gain an additional 40%-50% of extracellular fluid, including blood volume, as pregnancy proceeds. Scott D E, “Anemia in pregnancy,” Obstet. Gynecol. Annu., 1972;1:219-244.

The present invention seeks to determine if preeclampsia could be an example of volume expansion-mediated hypertension. A review of the literature revealed a dearth of animal models, in general, and no information on the possibility that at least some forms of preeclampsia (a syndrome, not a single disease process) could be related to excessive volume expansion. Furthermore, elevated levels of marinobufagenin (“MBG”) have been reported in preeclamptic patients. Gonick H C, Ding Y, Vaziri N D, et al., “Simultaneous measurement of marinobufagenin, ouabain, and hypertension-associated protein in various disease states,” Clin. Exp. Hypertens., 1998;20:617-627 and Lopatin D A, Ailamazian E K, Dmitrieva R I, et al., “Circulating bufodienolide and cardenolide sodium pump inhibitors in preeclampsia,” Am. J. Hypertens., 1999;17:1179-1187. Volume expansion is known to stimulate MBG secretion and elaboration. Fedorova O V, Doris P A, Bagrov A Y, Endogenous marinobufagenin-like factor in acute plasma volume expansion,” Clin. Exp. Hypertens., 1998;20:581-591 and Bagrov A Y, Fedorova O V, Dmitrieva R I, et al., “Plasma marinobufagenin-like and ouabain-like immunoreactivity during saline volume expansion in anesthetized dogs,” Cardiovasc. Res., 996;31:296-305. It has been determined that MBG is produced both in the adrenal glands (Dmitrieva R I, Bagrov A Y, Lalli E, et al., “Mammalian bufadienolide is synthesized from cholesterol in the adrenal cortex by a pathway that is independent of cholesterol side-chain cleavage,” Hypertension, 2000;36:442-448 and Lichtstein D, Steinitz M, Gati I, et al., “Bufodienolides as endogenous Na, K-ATPase inhibitors: biosynthesis in bovine and rat adrenals,” Clin. Exp. Hypertens., 1998;20:573-579) and in the placenta (Hilton P J, White R W, Lord G A, et al., “An inhibitor of the sodium pump obtained from human placenta,” Lancet, 1996;348:303-305), and perhaps, also in other sites in the body, e.g., the hypothalamus. Additionally, MBG is a known vasoconstrictor that can cause hypertension. Schoner, W., “Endogenous cardiac glycosides, a new class of steroid hormones,” Eur. J. Biochem., 2002;269:2240-2448. It is also a cardiac inotrope.

Despite the foregoing knowledge, there remains a very real and substantial need to develop an effective means for ascertaining, with accuracy, and repeatability the amount of marinobufagenin in a body specimen, such as urine or blood.

### SUMMARY OF THE INVENTION

The present invention has met the above-described need by providing an effective assay for determining, with precision, the amount of marinobufagenin in a body specimen to thereby facilitate an accurate evaluation of whether an individual has hypertension. The methods of the present invention also involve the use of unique compounds which facilitate such a determination of marinobufagenin concentration in a body specimen.

It is an object of the present invention to employ an accurate method for determining the amount of marinobufagenin in a body specimen in order to facilitate an evaluation regarding whether an individual is suffering from hypertension.

It is a further object of the method of the present invention to employ certain unique compounds which may be used to create conjugates for use in such method.

It is a further object of the present invention to provide a method which employs marinobufagenin linked to a protein as an immunogenic conjugate to generate polyclonal rabbit and murine monoclonal antibodies (“mab”) which may be employed in determining quantitatively the amount of marinobufagenin in a body specimen.

It is yet another object of the present invention to provide such a method which may employ an ELISA test in effecting determination of the marinobufagenin concentration.

It is yet another object of the present invention to provide a method of accurately determining the marinobufagenin concentration in a body specimen in order to ascertain whether volume expansion-mediated hypertension exists in a patient.

It is yet another object of the present invention to provide such a method for determining the quantity of marinobufagenin in a body specimen, so as to facilitate an accurate determination regarding whether essential hypertension exists in order to facilitate an appropriate course of treatment for the patient.

These and other objects of the present invention will be more fully understood in the following detailed description of the invention on reference to the illustrations appended hereto.

### BRIEF DESCRIPTION OF THE DRAWINGS

FIG. 1 illustrates the structure of marinobufagenin.

FIG. 2 illustrates the structure of resibufogenin.

FIG. 3 illustrates the structure of cinobufotalin (“CINO”).

FIG. 4 illustrates the structure of a prior art marinobufagenin-conjugate immunogen, as used in previous, fundamentally-different approaches which do not expose the difference between marinobufagenin and resibufogenin (“RBG”).

FIG. 5 illustrates an immunogen derived from cinobufotalin.

FIG. 6 shows the synthesis of a novel bovine serum albumin (BSA)-marinobufagenin immunogen 5 obtained from cinobufotalin 3.

FIG. 7 shows the structure of a novel β-lactoglobulin (BLG)-marinobufagenin conjugate 11 from cinobufotalin 3 (“CINO3”).

FIG. 8 shows a biotin-mediated ELISA format. This ELISA was used for the identification of monoclonal anti-marinobufagenin antibodies with high intrinsic affinity. An alternative ELISA format is shown in FIG. 11.

FIG. 9 shows a reaction for the synthesis of marinobufagenin (“MBG”) to marinobufagenin-biotin conjugate 13 from CINO-ester conjugate 10.

FIG. 10 illustrates a synthesis for conversion of CINO3 with intermediate 7 derived from CINO (see FIG. 6).

FIG. 11 shows the screening ELISA used for the detection of antibodies against marinobufagenin. Initially, both BSA and BLG were employed as the carrier protein. In the former case, excess BSA was added to the reaction mixture to inhibit anti-BSA antibodies from binding to the conjugate. When the BLG-marinobufagenin congugate became available, BLG-marinobufagenin was used for antibody detection. This ELISA format was used as the basis for a competitive ELISA for the detection of marinobufagenin in body specimens.

### DESCRIPTION OF THE PREFERRED EMBODIMENTS

As employed herein, the term “body specimen” refers to blood, urine, or tissue extracts from a human being.

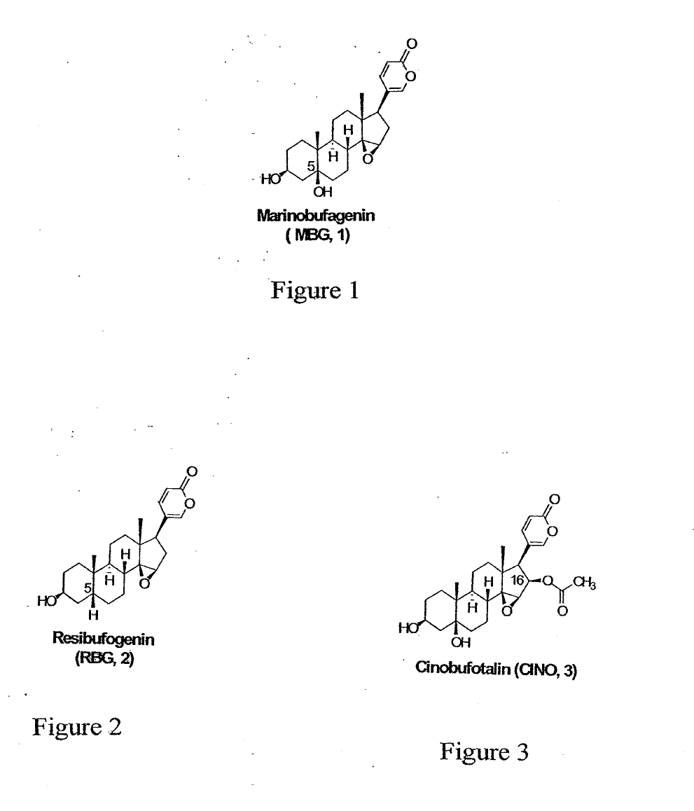

Marinobufagenin (FIG. 1) is a steroid. As steroids are antigenic but not immunogenic, the focus of the present method is to chemically modify marinobufagenin in order to make it immunogenic by conjugating it to a carrier protein, such as bovine serum albumin (“BSA”), for example. In a preferred form, the present method modifies the marinobufagenin molecule chemically. This consists of attaching a linker, which is then attached to bovine serum albumin or another suitable protein to make it immunogenic. The introduction (injection) into mice and rabbits of this immunogenic compound has successfully caused production of antibodies recognizing marinobufagenin. Balb/c mice are preferred for the production of monoclonal antibodies because of the availability of suitable fusion partners (murine myeloma cell lines) for the development of hybridomas, which are the cell lines that produce monoclonal antibodies. Monoclonal antibodies can be more effective for purposes of the present method owing to their remarkable specificity. By contrast, polyclonal antisera tend to have superior affinity, which is reflected in increased sensitivity of resulting immunoassays.

Any of a number of suitable linkers bearing CH₂, C(O), O, or N atoms can be employed to provide an appropriate tether length between the hapten and the carrier protein or the hapten and biotin to enable generation of an immune response and generate antibodies or for use as a tracer (=labeled antigen) in the ELISA assay, respectively. The ranges given refer to the number of atoms that separate the hapten and the carrier protein or biotin. The linkers employed preferably include CH₂, C(O), O, or N atoms and can vary in length from 5 to 20 atoms but preferably are 7 to 12 atoms in length.

It is an object of the present invention to provide sensitivity of the method of the invention to the desired level of pg/ml (picograms per milliliter). The method of the present invention preferably employs an ELISA assay which may be relatively standard, but which is unique in the chemical process it employs for attaching marinobufagenin to a protein. The design of the conjugate is also reflected in the specificity of the antibodies which are produced, i.e., their capacity to discriminate almost perfectly between MBG and RBG.

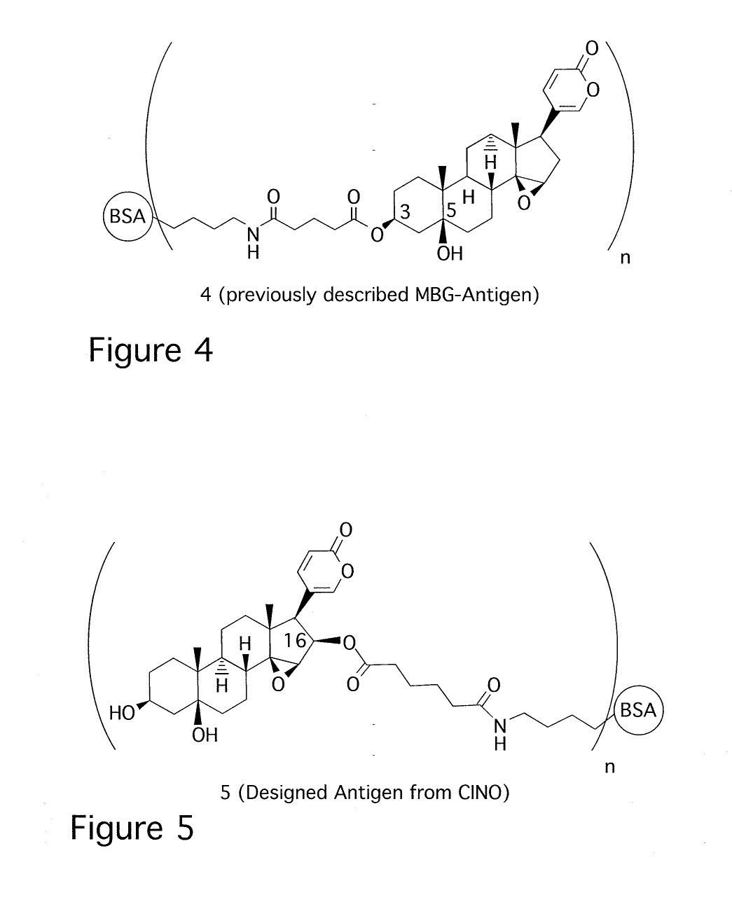

As a first step in the present process, marinobufagenin derivatives are readied for conjugation to proteins. An antibody to MBG was previously generated using an antigen prepared directly from MBG (FIG. 1) (Yoshika M, Komiyama Y, Konishi M, et al., “Novel Digitalis-Like Factor, Marinobufotoxin, Isolated From Cultured y-1 Cells, and Its Hypertensive Effect in Rats,” Hypertension, 2007;49:209-214). The antigen 4 was prepared by attachment of a 5-carbon linker to the C3-hydroxyl group of MBG using glutaric anhydride (FIG. 4).

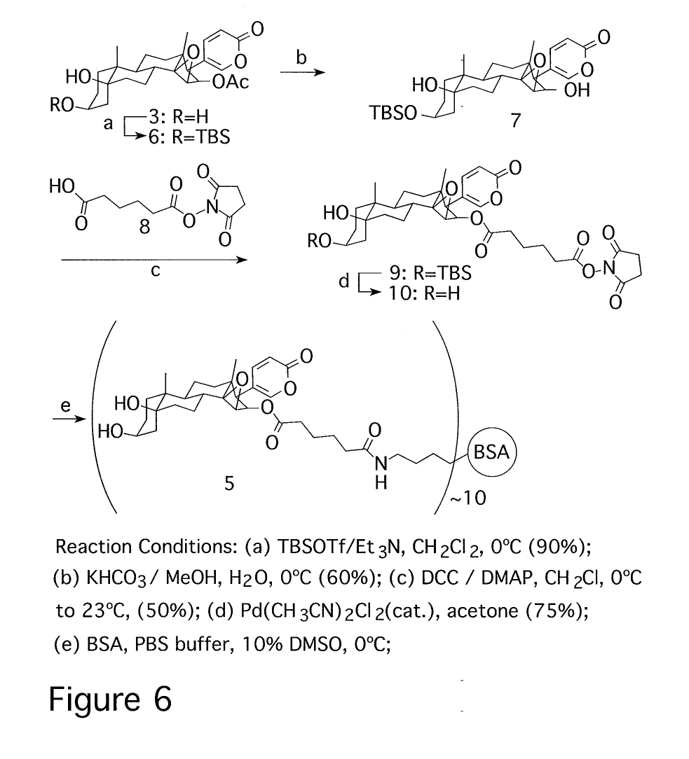

The derived carboxyl acid derivative was then coupled to the surface lysine groups of the carrier protein bovine serum albumin (“BSA”) by the activated N-hydroxysuccinimidyl ester method (Hosoda H, Sakai Y, Yoshida H, et al., “The Preparation of Steroid N-hydroxysuccinimide Esters and Their Reactivities with Bovine Serum Albumin,” T. Chem. Pharm. Bull., 1979;2:742-746). While the generated antibodies exhibited low cross-reactivity with some other bufodienolide natural products and cardenolides 6 (FIG. 6), there were no data for resibufogenin 2 (“RBG,” FIG. 2), a compound of great interest. Given that the only difference between RBG and MBG is the absence of the C5-hydroxyl group, it is challenging to produce an antibody that will recognize MBG with minimum cross-reactivity to RBG, especially when the point of attachment of marinobufagenin to the carrier protein is close to this site of difference, e.g., the C3-hydroxyl. The present invention provides the design and synthesis of an immunogen MBG antigen 5 (FIG. 5) with an alternative point of attachment to minimize potential cross-reactivity to RBG.

The next step is the synthesis of immunogenic bovine serum albumin (“BSA”)-marinobufagenin and β-lactoglobulin (“BLG”)-marinobufagenin conjugates. To develop an antibody with high specificity and affinity, the excellent potential utility of a commercially available bufodienolide, cinobufotalin 3 (CINO, FIG. 3) for antigen synthesis was exploited. This plant-derived natural product is very closely related structurally to MBG only differing by an additional hydroxyl group at C16 modified as an acetate group. Thus, CINO provides a viable attachment point for a carrier protein by a linker attached to the C16-hydroxyl once the acetate has been removed (FIGS. 4 and 5). CINO maintains all structural features of MBG including the critical C5-hydroxyl group, which is the only functional group that distinguishes it from RBG. Most importantly, the alternative point of attachment, namely the C16-OH of CINO versus the C3-OH of MBG (FIG. 6), extends the distance between the linker and the critical C5-hydroxyl group which was expected to increase the probability of generating antibodies that recognize this subtle difference between marinobufagenin and resibufogenin. The CINO-NHS ester 10 as shown in FIG. 6 was synthesized. CINO was first protected as the C3 silyl ether 6. Subsequent deacetylation under mild basic conditions revealed the C16-hydroxyl as a handle for conjugation to carrier proteins. The carboxylic acid-NITS ester 8 was coupled to the C16-hydroxyl of 7 (FIG. 6) under standard conditions. Deprotection of the silyl ether at C3 provided an MBG hapten 9 (FIG. 6) that bears an activated ester readied for coupling to carrier proteins. This synthesis is readily scaled to provide 10's of milligrams of the NHS ester-activated MBG-hapten 10 which was used to conjugate to both BSA and, also, BLG.

### Example I

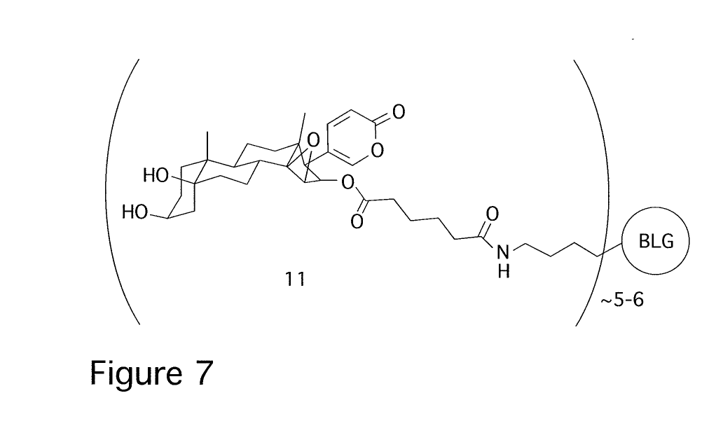

An example of the preparation of the BSA-marinobufagenin antigen will be considered. To prepare the BSA-MBG antigen, the hapten 9 (FIG. 6) was reacted with the surface lysines of BSA by first dissolving the activated ester in 100 μL of dimethyl sulfoxide (“DMSO”) and then mixing with a 1 mL solution of BSA in phosphate-buffered saline (“PBS”) buffer (pH =7.2; 0.1 M Phosphate+0.1 mM NaCl; 10mg/mL) in an Eppendorf tube. The mixture was thoroughly stirred and left at 23° C. for 3-4 hours. A 20 μL aliquot was analyzed by MALDI mass spectrometry to verify successful conjugation. Mass-spectrometric comparison of the native carrier protein (“BSA”) and the resulting BSA-MBG conjugate 5 (FIG. 5) indicated that the MBG hapten 9 (FIG. 6) was conjugated on average to BSA in a ˜10:1 molar ratio; that is, an average of ˜10 molecules of MBG to every molecule of protein. In a similar manner, a second conjugate was prepared based on an alternative carrier protein, β-lactoglobulin (“BLG”) that was used to coat ELISA plates and measure MBG immunoreactivity without the interference of anti-BSA antibodies that are part of the antiserum against the MBG-BSA immunogen (FIG. 7). In this case, preliminary mass spectrometry data suggested a loading of 5˜6 molecules of the MBG-hapten to every molecule of BLG.

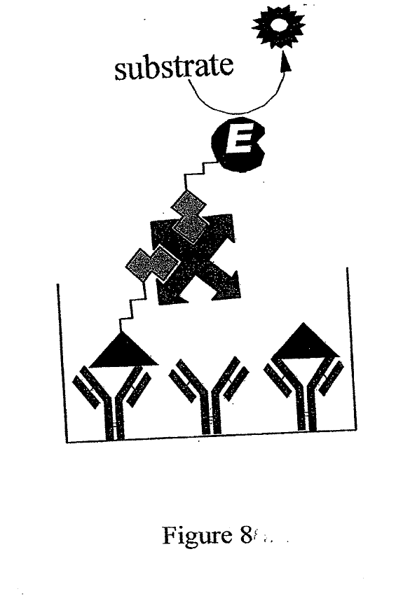

ELISA strategies, other than directly binding MBG to protein, serve as a coating reagent for ELISA plates. The other possible format of competitive ELISA for a small molecule like MBG is that the tracer is biotinylated MBG. FIG. 8 shows the biotin-mediated ELISA. An advantage of the format of FIG. 8 is that it offers more flexibility to choose just about any endpoint one can imagine, because the binding protein for biotin avidin is commercially available in a broad variety of labeled forms. It would, for example, be an easy way to switch over to fluorescence, or time-resolved fluorescence, as the readout for the immunoassay. Another important application of the MBG-biotin conjugate is that it can be used to affinity purify the cellular protein target of MBG.

### Example II

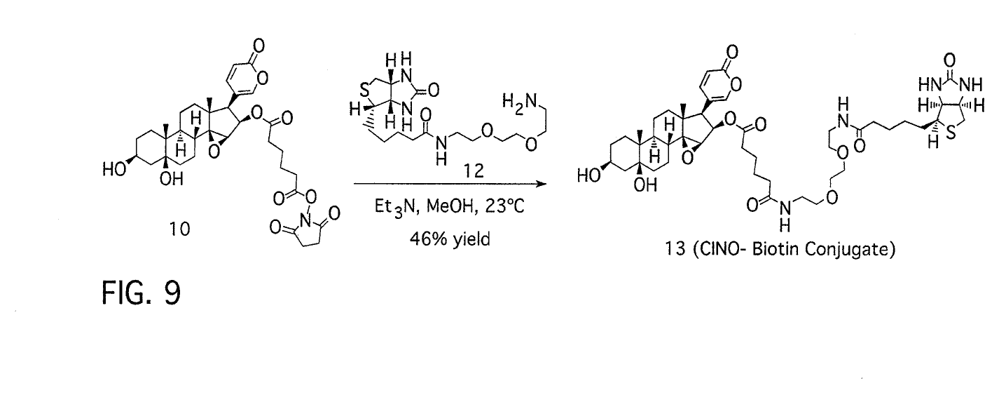

An example of the preparation of the marinobufagenin (CINO)-biotin conjugate 13 will be considered. To prepare the MBG (CINO)-biotin conjugate 13, the NHS ester activated MBG hapten 10 (FIG. 9) derived from cinobufatolin (CINO) was mixed with a commercially available (+)-biotin-(PEO)₃-amine in the presence of Et₃N. After stirring at 23° C. for 24 hours, the mixture was purified by flash chromatography to give the marinobufagenin CINO-biotin conjugate in 46% yield.

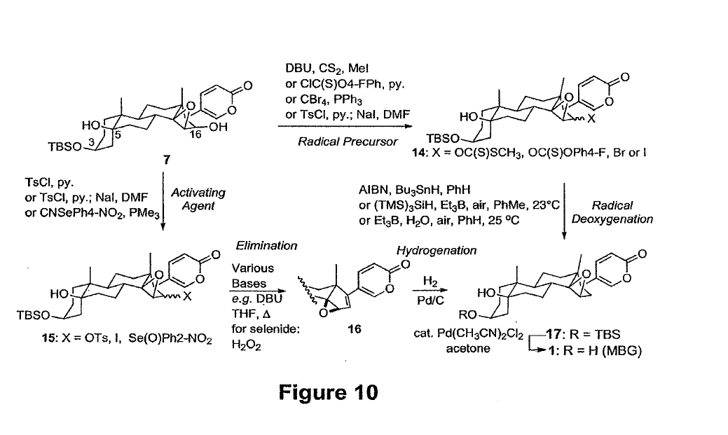

In order to “scale-up” the assay, so that large numbers of samples can be analyzed, synthesis of MBG will be required as a control for the assay. This may be accomplished by converting cinobufotalin (“CINO”) to MBG by a deoxygenation process. There are several mild methods which can be employed, and given the ready availability of CINO, a short sequence for deoxygenation of CINO to deliver 10's of milligrams of MBG can be provided. FIG. 10 outlines two routes that can be followed for the conversion of CINO to MBG. The methods may include mild radical deoxygenation conditions and an elimination/hydrogenation sequence.

While there are several methods for radical deoxygenation including those pioneered by Barton (Hartwig W., “Modern Methods for the Radical Deoxygenation of Alcohols,” Tetrahedron, 1983;39:2609-2645), a more recent use of tris(trimethylsilyl) methane (TMS)-(CH) as a tin-free reducing agent by Perchyonok (Perchyonok V T, “On the Use of (TMS) 3CH as Novel Tin-Free Radical Reducing Agent,” Tetrahedron Letters, 2006;47:5163-5165), and mild conditions reported by Wood will also be explored (Spiegel D A, Wiberg K B, Schacherer L N, et al., “Deoxygenation of Alcohols Employing Water as the Hydrogen Atom Source,” J. Am. Chem. Soc., 2005;127:12513-12515). These processes typically involve conversion to a suitable radical precursor 14 (FIG. 10) followed by radical initiated deoxygenation/reduction, and in this case, would deliver C3-TBS MBG 17. A deprotection step previously developed for MBG hapten synthesis (FIG. 6) will be utilized for the final deprotection to deliver MBG 1 (FIG. 1). A second strategy involves standard activation of the C16 hydroxyl to provide derivatives 15 and elimination to provide olefin 16, which will then be hydrogenated to deliver MBG following TBS deprotection. Elimination can proceed via the tosylate or the iodide (15, X=Ts, I respectively, FIG. 10) followed by elimination with non-nucleophilic bases, such as diazabicycloudecane (DBU). Another elimination method involves introduction of the selenide (15, X=Se(O) Ph2-NO2, FIG. 10) followed by oxidation leading to direct syn-elimination of the selenoxide by the method of Grieco (Grieco P A, Sydney, Gilman, et al., “Organoselenium Chemistry. A facile one-step synthesis of alkyl aryl selenides from alcohols,” J. Org. Chem., 1976;41(8):1485-1486). These elimination processes will provide alkene 16 (FIG. 10) which can then be subjected to hydrogenation as described by Meinwald in related systems (Liu Z, “Meinwald J. 5-(Trimethylstannyl)-2H-pyran-2-one and 3-(Trimethylstannyl)-2H-pyran-2-one: New 2H-Pyran-2-one Synthons,” J. Org. Chem., 1996;61:6693-6699) to deliver C3-TBS MBG 17 (FIG. 10) with the correct stereochemistry at C17 based on this precedent. Final deprotection as described herein will deliver MBG.

### Example III

The ELISA assay involved the following:

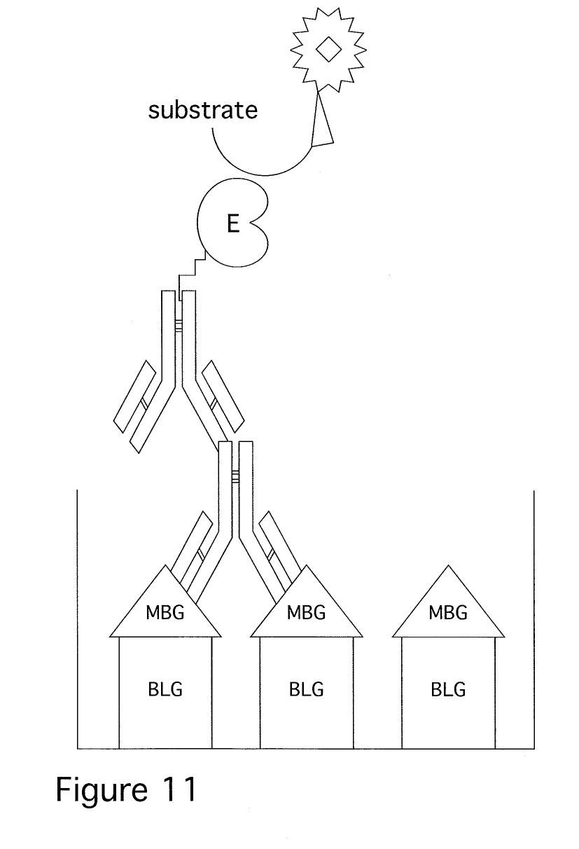

Four Balb/c mice were immunized six times with gradually decreasing doses of the BSA-MBG conjugate (FIG. 6) emulsified in RIBI adjuvant, in order to promote affinity maturation of the immune response. The titer of anti-MBG antibodies was monitored by testing the mice sera in an ELISA against the BSA-MBG conjugate, after blocking the anti-BSA antibodies with an excess of BSA (FIG. 11). Bound anti-MBG antibodies were detected using a peroxidase-conjugated anti-mouse IgG and an appropriate substrate system. Following the last immunization step, the mice were sacrificed and their splenocytes were harvested. Splenocytes from the mouse with the best anti-MBG titer were fused with Sp2/0 myeloma cells in a 3:1 ratio by electrofusion, and the resulting hybridomas were drug selected by HAT medium. Two weeks post-fusion, one hundred different hybridomas were selected (based on the same ELISA as shown in FIG. 11) for expansion and cryogenic storage. These hybridomas produced specific anti-MBG antibodies, as ascertained by a maximal anti-MBG ELISA response, as well as more or less complete inhibition of the obtained signal by addition of excess MBG. The one hundred best hybridomas were selected, expanded, and cryogenically stored.

In order to narrow the number of cell lines to a manageable shortlist of monoclonal antibodies (“mabs”) with the highest affinity for MBG, a two-step process was followed.

In a first step (results not shown), each of the hybridoma supernatants were assayed for their mouse IgG content. By knowing the exact antibody content, a comparison of all antibodies for their tracer binding capacity at identical antibody concentration could be made.

In the second step of the process, each monoclonal antibody (“mab”) could be incubated at two different concentrations (1 ng/ well and 0.1 ng/well, respectively) with the same concentration of biotinylated MBG referred to as “the tracer;” see FIG. 8. The amount of tracer was 1 ng per well. The results of this tracer binding assay showed that about ten mabs were able to bind substantial amounts of tracer at a concentration of 0.1 ng mab/well. Those ten primary lines were brought back in culture out of cryogenic storage and were cloned by limiting dilution, i.e., every line needed to be re-grown from 1 cell per well to ensure that it is truly monoclonal. This is desirable to avoid oligoclonal cell mixtures that might contain non-secreting cells that would overgrow the secretors, or the presence of two different antibody-secreting lines in one well, an equally undesirable situation.

This growth process takes about 2 weeks before there are again enough cells to test by ELISA which subclones are monoclonal and secrete the desired mab. Once this has been determined, it takes about another 2 weeks to expand these subclones to where they are cryogenically stored again (multiple freezers and multiple copies). This was followed by testing which of those mabs yielded the most sensitive assay using the assay format shown in FIG. 8.

In addition to synthesizing the marinobufagenin antigens and conjugating them with proteins in order to effectively induce an immune response antibody action, included within the present invention are the compounds designated 5 (FIG. 5), 6 (FIG. 6), 7 (FIG. 6), 9 (FIG. 6), 10 (FIG. 6), 11 (FIGS. 9), and 13 (FIG. 9).

In a preferred practice of the invention, the antibodies produced may be stored in frozen condition until such time as they are to be used, at which time they may be thawed and employed in the ELISA test.

In an alternative approach, rabbits were immunized with the BSA-MBG conjugate for the production of polyclonal antisera. Six New Zealand White rabbits were immunized with the BSA-MBG conjugate according to routine lab protocols using decreasing immunogen doses in order to promote affinity maturation. The resulting polyclonal sera were assessed for the presence of MBG-specific antibodies by ELISA (FIG. 11). In this particular format, two of the rabbits produced a strong humoral immune response that allowed dilution of the primary (rabbit) antiserum down to 1 in 4*10⁶, even when using a relatively insensitive photometric readout. In addition, competition assay with a panel of more or less related compounds of interest showed that only RBG showed any cross-reactivity with our antisera, which was only measurable at extremely high doses. This particular assay has now been equipped with a chemifluorescent readout (using commercially available reagents) and has now reached the target sensitivity of less than 10 pg MBG/ml. As expected, rat urine samples can be measured without prior treatment, but rat plasma and/or serum samples require solid phase extraction using a C8 solid phase and acetonitrile in the mobile phase. Upon drying the eluate of obtained from the extraction cartridge by vacuum centrifugation, MBG can be measured in the circulation of the rat.

### Example IV

Four Balb/c mice were immunized six times with gradually decreasing doses of the BSA-MBG conjugate, in order to promote affinity maturation of the immune response. The conjugate was emulsified in RIBI adjuvant [1 mg Lipid A, monophosphorylated from E. coli F583 (Sigma), 1 mg Trehalose 6, 6′-dimycolate from Mycobacterium tuberculosis (Sigma, S t. Louis, M O), 0.4% (v/v) Tween-80 (Sigma), 4.5% (v/v) squalene in 2 mL MQ-water] prior to immunization. The titer of anti-MBG antibodies was monitored by testing the mice sera in an ELISA against BSA-MBG, after blocking the anti-BSA antibodies with an excess of BSA. Bound anti-MBG antibodies were detected using a peroxidase-conjugated anti-mouse IgG and the TMB substrate system. Following the last immunization step, the mice were sacrificed and their splenocytes were harvested. Splenocytes from the mouse with the best anti-MBG titer were fused with Sp2/0 myeloma cells (ATCC, Manassas, Va.) by electrofusion. Splenocytes and myeloma cells were counted, and the number of cells needed to achieve a splenocyte:myeloma ratio of 3:1 was determined. Splenocytes and myeloma cells were then mixed and treated with dispase (EMD Biosciences, San Diego, Calif.) at 10 μg/ml, for 5 min at room temperature. Cells were washed three times with electrofusion buffer (0.255 M sucrose, 0.2 mM CaCl₂, 0.2 mM MgCl₂, sterile-filtered), then resuspended in 0.5 ml electrofusion buffer, and gently spread on the electrofusion slide. The electrofusion chamber (Meander Fusion Chamber, Harvard Apparatus, Holliston, Mass.) was then connected to the Electro Cell Manipulator® ECM 2001 (Harvard Apparatus). Using an AC current at 6V and for 30 sec, the cells were first aligned in a pearl line fashion. One high-voltage pulse (DC current at 60V, 15 μsec) then caused adjacent cells to fuse. Following fusion, cells were allowed to sit in the chamber for 30 min at room temperature, and were then transferred to a reservoir containing fusion medium (Complete DMEM growth medium, Mediatech, Manassas, Va.) with addition of cytokines to sustain single cell growth. Cells were gently mixed into the medium, and transferred into two 96-well plates (Nunc, Rochester, N.Y.). To eliminate non-fused myeloma cells, selection with HAT media supplement (Sigma) was then applied for seven days. Hybridoma cells were maintained in Complete DMEM growth medium containing HT media supplement (Sigma). Two weeks post-fusion, one hundred different hybridomas were selected for expansion and cryogenic storage. These hybridomas produced specific anti-MBG antibodies, as ascertained by a maximal anti-MBG ELISA response, as well as by partial to complete inhibition of the obtained signal by addition of excess MBG.

Whereas particular embodiments of the present invention have been described herein for purpose of illustration, it will be evident to those skilled in the art that numerous variations of the details may be made without departing from the invention as set forth in the appended claims.

What is claimed is:

1. A method of synthesizing marinobufagenin immunogen comprising conjugating marinobufagenin to a protein by means of a linker.

2. The method of claim 1, including employing a 5-carbon linker and securing it to the C3-hydroxyl group of said marinobufagenin.

3. The method of claim 2, including employing bovine serum albumin as said protein.

4. A marinobufagenin immunogen comprising the structure of FIG. 5.

5. A method of synthesizing the marinobufagenin immunogen of FIG. 5 employing the reaction of FIG. 6.

6. The compound of the first reactant shown in FIG. 6, wherein R=tert-butyldimethylsilyl.

7. An intermediate compound employable in a method of making the marinobufagenin immunogen of FIG. 5 comprising the intermediate compound designated 7 in FIG. 6.

8. An intermediate compound employable in a method of making the marinobufagenin antigen of FIG. 5 comprising the intermediate compound designated 9 in FIG. 6 where R=tert-butyldimethylsilyl.

9. A compound employable in the making of a marinobufagenin conjugate comprising the compound designated 10 in FIG. 6.

10. A compound employable in the method making of marinobufagenin conjugate comprising the compound designated 11 of FIG. 9.

11. A compound employable in the making of a marinobufagenin conjugate comprising the compound designated 5 in FIG. 5.

12. A compound employable in the making of a marinobufagenin conjugate comprising the compound designated 6 in FIG. 6.

13. A compound employable in the making of a marinobufagenin conjugate comprising the compound designated 13 in FIG. 9.

14. A method of making CINO-biotin conjugate comprising the reaction shown in FIG. 9.

15. The method of claim 1, including employing a 7 to 12 carbon linker as said linker.

16. The method of claim 1, including said linker having atoms selected from the group consisting of CH₂, C(O), O, and N atoms.

17. The method of claim 1, including said protein being selected from the group consisting of bovine serum albumin and β-lactoglobulin.

18. A method of determining the marinobufagenin concentration in a body specimen, including obtaining antibodies by creating an immunogen by securing marinobufagenin to a protein by means of a linker and subsequently introducing the immunogen into a host to generate responsively the desired antibodies.

19. The method of claim 18, including employing an ELISA test using said antibodies to determine marinobufagenin concentration in a body specimen.

20. The method of claim 19, including selecting said body specimen from the group consisting of urine, blood, and tissue.

21. The method of claim 18, including employing said antibodies to determine marinobufagenin concentration in a body specimen.

22. The method of claim 18, including said antibodies being selected from the group consisting of monoclonal antibodies and polyclonal antibodies.

23. The method of claim 22, including said antibodies being monoclonal antibodies.
